(19) United States
(12) Patent Application Publication
(10) Pub. No.: US 2026/0297674 A1
(43) Pub. Date: Oct. 1, 2026

(54) COMPOSITIONS AND METHODS FOR DETECTING AND TREATMENT OF GLOMERULAR DISEASES

(71) Applicant: The Texas A&M University System, College Station, TX (US)

(72) Inventors: Mary B. Nabity, College Station, TX (US); Ariana D. Cherry, Bryan, TX (US); Candice P. Chu, Bala Cynwyd, PA (US); Jessica A. Hokamp, Bryan, TX (US)

(21) Appl. No.: 19/479,965

(22) PCT Filed: May 9, 2024
(86) PCT No.: PCT/US2024/028545

§ 371 Date: Oct. 29, 2025

Related U.S. Application Data
(60) Provisional application No. 63/502,534, filed on May 16, 2023.

(51) Int. Cl. C12Q 1/6883; C12Q 1/6806; C12Q 1/6825; C12Q 1/686

(52) U.S. Cl. CPC C12Q 1/6883; C12Q 1/6806; C12Q 1/6825; C12Q 1/686; C12Q 2600/106; C12Q 2600/112; C12Q 2600/178

(57) ABSTRACT

Disclosed are methods and compositions for detecting glomerular disease in dogs and effectively treating dogs with glomerular diseases such as amyloidosis, glomerulosclerosis, or immune complex-mediated glomerulonephritis. miRNAs associated with glomerular disease are also provided, as are kits, and methods of use.

### REFERENCE TO RELATED APPLICATIONS

This application claims the benefit of U.S. provisional application No. 63/502,534, filed May 16, 2023, herein incorporated by reference in its entirety.

### INCORPORATION OF SEQUENCE LISTING

The sequence listing that is contained in the file named “TAMC077WO_ST26.xml,” which is 8.11 kilobytes as measured in Microsoft Windows® operating system and was created on May 7, 2024, is filed electronically herewith and incorporated herein by reference.

### FIELD OF THE INVENTION

The present invention relates generally to the field of veterinarian sciences. More specifically, the invention relates to methods and compositions for detecting glomerular disease and treating the same.

### BACKGROUND OF THE INVENTION

Chronic kidney disease (CKD) is a significant cause of morbidity and mortality in all breeds of dogs, and it is commonly caused by underlying glomerular diseases. The three most common glomerular diseases in dogs with naturally occurring chronic kidney disease (CKD) are amyloidosis (AMYL), glomerulosclerosis (GS), and immune complex-mediated glomerulonephritis (ICGN). Each of these glomerular diseases has different treatment and prognostic considerations.

Proper treatment relies on an accurate diagnosis of the type of glomerular disease. However, there are currently no non-invasive or minimally-invasive tests to reliably diagnose the category of glomerular disease. Renal biopsy and comprehensive pathologic examination are currently required for diagnosing specific glomerular diagnostic categories and guiding proper treatments. However, such procedures are not undertaken for the majority of dogs because the patient is either deemed unsuitable for anesthesia and/or the cost of the renal biopsy is prohibitive. Despite the current treatment recommendations, there is a lack of evidence-based therapies that take into account the category of glomerular disease. Furthermore, without the aid of a renal biopsy, current treatment recommendations may rely on incorrect assumptions and lead to unintentional negative outcomes. Thus, there remains a need to provide methods and compositions for accurate detection paired with targeted treatment of glomerular disease, especially in companion animals such as dogs and cats.

### SUMMARY OF THE INVENTION

One aspect of the present invention provides a method of detecting glomerular disease in a dog comprising obtaining a sample from the dog that comprises at least a first miRNA associated with glomerular disease; detecting in the sample the presence of an elevated level of the miRNA relative to a control dog that lacks glomerular disease; wherein the elevated level of the miRNA relative to the control is indicative of glomerular disease in the dog. In one embodiment, the glomerular disease comprises amyloidosis, glomerulosclerosis, or immune complex-mediated glomerulonephritis. In particular embodiments, the sample is selected from the group consisting of serum, plasma, whole blood, and urine. In certain embodiments, such methods comprise detecting in the sample an elevated level of at least a second miRNA; or detecting in the sample an elevated level of miR-126, miR-128, miR-21, miR-182, or miR-335. In certain embodiments, the elevated level of the miRNA indicates the stage of glomerular disease progression. In further embodiments, the elevated level of the miRNA further indicates fibrosis severity. In still other embodiments, the elevated level of the miRNA indicates that the dog has late stage glomerular disease. In specific embodiments, detecting in the sample the presence of an elevated level of the miRNA comprises RNA-Seq analysis or qRT-PCR analysis.

In another aspect, the invention provides a method of treating a dog with glomerular disease comprising obtaining a sample from the dog that comprises at least a first miRNA associated with glomerular disease; detecting in the sample the presence of an elevated level of the miRNA relative to a control dog that lacks glomerular disease; and treating the dog with glomerular disease based on the elevated level of the miRNA relative to the control. In some embodiments, treating the dog comprises no treatment. In other embodiments, treating the dog comprises immunosuppressive therapy; or angiotensin-converting enzyme (ACE) inhibitors (e.g., enalapril, benazepril, captopril, ramipril), angiotensin receptor blockers (ARBs; e.g., telmisartan, losartan), managing of blood pressure (e.g., beta-blockers, calcium channel inhibitors), anticoagulants (e.g., aspirin, clopidogrel, rivaroxaban), omega-3 fatty acid supplementation, or use of specially formulated diets for renal disease patients. In specific embodiments, the immunosuppressive therapy comprises mycophenolate, cyclophosphamide, azathioprine, chlorambucil, or cyclosporine.

In yet another aspect, the invention provides a method of detecting the presence of a miRNA corresponding to glomerular disease in a sample, the method comprising contacting the sample comprising miRNA with a reverse transcriptase, wherein said reverse transcriptase produces a cDNA sequence corresponding to the miRNA; and performing a polynucleic acid amplification reaction, thereby producing an amplicon; and detecting said amplicon, wherein said amplicon comprises a DNA sequence produced from SEQ ID NO:1, SEQ ID NO:2, SEQ ID NO: 3, SEQ ID NO:4, or SEQ ID NO:7. In certain embodiments, the method further comprises quantifying the amplicon in the sample.

Another aspect of the invention comprises a kit comprising: a miRNA primer pair; a means to quantitatively measure at least one miRNA; and a container for the substrate. In certain embodiments, the miRNA primer pair quantitatively measures a miRNA selected from the group consisting of: miR-126, miR-128, miR-21, miR-182, and miR-335. The kit may further comprise a substrate comprising a detectable label. In certain embodiments the detectable label is a fluorescent, radioactive, chromogenic, or chemiluminescent tag.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Clinical parameters across diagnostic categories from dogs used in RNAseq and qRT-PCR. Boxplots of age, serum creatinine (sCr), and urine protein:creatinine ratio (UPC) distribution for dogs whose samples were used for the small RNA-seq analysis (Panel A, Panel C, Panel E) versus the qRT-PCR dataset (Panel B, Panel D, Panel F) among different diagnostic categories. The median expression and individual values are displayed on the boxplot; bars represent the upper, middle, and lower quartiles. The Mann Whitney U test was used in a pair-wise manner to compare the median of each category. * P<0.05; ** P<0.01; *** P<0.001. AMYL: amyloidosis, GS: glomerulosclerosis, and ICGN: immune complex-mediated glomerulonephritis.

FIG. 2. Differentially expressed (DE) circulating and urinary miRNAs identified in CKD dogs (based on glomerular disease category) versus controls. Dogs with CKD were compared to controls regardless of stage (Panel A, Panel D), in Stage A (Panel B, Panel E), and Stage B (Panel C, Panel F). The total number of DE miRs are shown in parenthesis. Only those DE miRs that were also differentially expressed when comparing all dogs with CKD with controls in the respective group are included. Differential expression was based on an absolute fold change >2 and an adjusted α-value <0.05. [S1: Stage A CKD; S2: Stage B CKD; AMYL (amyloidosis), GS (glomerulosclerosis) and ICGN (immune complex-mediated glomerulonephritis); ▴: upregulation in affected dogs, ▾: downregulation in affected dogs).

FIG. 3. Heatmap and clustering analysis of urinary miR-126, miR-335, and miR-128 in dogs with glomerular diseases and controls. Stage B dogs were included in this analysis. The expression levels of miR-126, miR-335, and miR-128 are significantly higher in azotemic dogs diagnosed with 12 (ICGN), G2 (GS), and A2 (AMYL), respectively. AMYL: amyloidosis, GS: glomerulosclerosis, and ICGN: immune complex-mediated glomerulonephritis.

FIG. 4. Normalized mean expression of urinary miR-126 based on qRT-PCR in clinically healthy dogs (Control n=14), dogs with amyloidosis (AMYL, n=14), glomerulosclerosis (GS n=19), and immune complex-mediated glomerulonephritis (ICGN n=19). The normalized mean expression of miR-126 was transformed to Log10 and plotted by diagnostic category. The median expression and individual values are displayed on the boxplot. Bars represent the upper, middle, and lower quartiles. Tukey for multiple testing was used to test for differential expression. * P<0.05; ** P<0.01; *** P<0.001.

FIG. 5. Normalized mean expression of urinary miRNA-486 (Panel A)-21 (Panel B), and -182 (Panel C) in clinically healthy (Control n=13), Stage A (n=21), and Stage B (n=32) dogs. The median expression and individual values are displayed on the boxplot. Bars represent the upper, middle, and lower quartiles. The Mann Whitney U test was used in a pair-wise manner to compare the median of each category. * P<0.05; ** P<0.01; *** P<0.001.

FIG. 6. Receiver operating characteristic (ROC) curve for miR-126 comparing dogs with immune complex-mediated glomerulonephritis (ICGN) versus dogs with amyloidosis (AMYL) and glomerulosclerosis (GS). The area under the curve (AUC) and its confidence interval are shown, using a cut-point of 1.48 Log10 miRNA expression, sensitivity is 90% (CI: 71-98%), and specificity is 92% (CI: 77-99%) for ICGN (P<0.001).

FIG. 7. Normalized read counts for urinary DE miRs identified between control, stage A and stage B CKD dogs using RNA-seq. Individual data points are plotted for each stage, and box plots show the median and upper and lower quartiles. Tukey for multiple testing was used (*P<0.05). Read counts were significantly lower for miR-486 (Panel A) and higher for miR-21 (Panel B), and miR-182 (Panel C) in Stage B dogs compared with Stage A dogs and, for miR-486, controls.

### BRIEF DESCRIPTION OF THE SEQUENCES

SEQ ID NO: 1 is the polynucleotide sequence of miR-126.

SEQ ID NO:2 is the polynucleotide sequence of miR-128.

SEQ ID NO:3 is the polynucleotide sequence of miR-21.

SEQ ID NO:4 is the polynucleotide sequence of miR-182.

SEQ ID NO:5 is the polynucleotide sequence of miR-151.

SEQ ID NO:6 is the polynucleotide sequence of miR-28.

SEQ ID NO:7 is the polynucleotide sequence of miR-335.

### DETAILED DESCRIPTION

Glomerular disease reduces the kidneys' ability to maintain a balance of certain substances in bloodstream. Normally, the kidneys filter toxins out of the bloodstream and excrete them in the urine but keep red blood cells and protein in the bloodstream. Glomerular disease is a common cause of chronic kidney disease (CKD) in dogs. For example, previous studies have shown that pathologic evidence of kidney disease is common in dogs, and it is often due to glomerular disease (Müller-Peddinghaus & Trautwein 1977; Macdougall 1986). The three most common glomerular diseases in dogs with naturally occurring chronic kidney disease (CKD) are amyloidosis (AMYL), glomerulosclerosis (GS), and immune complex-mediated glomerulonephritis (ICGN).

While overall prevalence of ICGN in dogs is unknown, data from the International Veterinary Renal Pathology Service (IVRPS) demonstrates that approximately half of dogs diagnosed with glomerular disease via a comprehensive renal evaluation have evidence of immune complexes (Schneider 2013; unpublished data). Based on this, ICGN is one of the most prevalent causes of glomerular disease in dogs. Furthermore, within the ICGN category are three major subcategories of disease: membranous glomerulonephritis (MGN), membranoproliferative glomerulonephritis (MPGN), and mesangioproliferative glomerulonephritis (MesGN).

Immunosuppressive therapy is recommended when there is pathologic evidence of active immune pathogenesis, most compellingly identified by electron dense deposits or definitive positive staining by immunofluorescence on a kidney biopsy (Segev 2013). These recommendations include several different treatment strategies depending on the severity of the disease, rate of progression, and co-morbidities (Segev 2013). However, only a small fraction of dogs with glomerular disease undergo a renal biopsy due to, e.g. concurrent disease(s) that place the dog at increased risk of anesthesia, concern about biopsy complications, and financial considerations. Clinicians often must make a clinical judgment whether or not to treat with immunosuppressive therapy in the absence of a kidney biopsy, but immunosuppressive therapy is contraindicated in non-ICGN glomerular diagnostic categories. Specifically, immunosuppressive therapy in non-ICGN glomerular disease can lead to gastrointestinal signs (e.g., anorexia, nausea, vomiting, diarrhea), bone marrow suppression, and secondary infections.

A non-invasive method for identifying ICGN would help ensure the correct treatment of ICGN related glomerular diseases as well as avoid the detrimental effects of contraindicated therapies in non-ICGN glomerular diseases. Moreover, the ability to non-invasively identify ICGN related glomerular disease could allow patients that are poor candidates for renal biopsy to be accurately diagnosed and treated effectively for the first time.

No published literature currently exists regarding the expression of miR-126 in canine or feline ICGN. The upregulation of miR-126 in ICGN thus serves as a novel means of ICGN detection. Moreover, the present disclosure provides methods of using such miRNAs to directly and accurately inform treatment decisions in canine or feline glomerular disease.

The present disclosure therefore represents a significant advance in the art in that it provides circulating and urinary miRNAs that may be used to differentiate between diagnostic categories of glomerular disease, and thus effectively treat such diseases. In particular, the present disclosure describes how the upregulation of urinary miR-126 was observed in dogs with ICGN compared to dogs with AMYL and GS. Thus, miR-126 represents a non-invasive biomarker to identify dogs for treatment with immunosuppressive therapy in the absence of a renal biopsy. Furthermore, the present disclosure also provides urinary miR-21 and miR-182 as markers of disease severity and fibrosis; and miR-335 and miR-128 as promising urinary biomarkers to categorize glomerulosclerosis and amyloidosis, respectively. The methods and compositions disclosed herein offer the opportunity to identify and effectively treat patients to improve and prolong quality of life without the need for a comprehensive renal biopsy.

Accordingly, provided herein are methods and compositions for identifying glomerular disease categories, primarily in dogs, that may be used to treat patients with the most effective therapy without relying on potentially incorrect assumptions. In some embodiments, the methods described herein are useful in the evaluation of a patient, for example, for evaluating diagnosis, prognosis, and response to treatment. In other aspects, the present invention comprises evaluating a dog with glomerular disease. In some embodiments, the evaluation may be selected from diagnosis, prognosis, and response to treatment.

Diagnosis refers to the process of attempting to determine or identify a possible disease or condition, such as, for example, a glomerular disease or specific category thereof such as ICGN, AMYL, and GS. Prognosis refers to predicting a likely outcome of a disease or condition, such as, for example, a glomerular disease or specific category thereof. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, improved renal function (e.g., decreased proteinuria, increased renal filtration), survival, and mitigation of proteinuric complications (e.g. hypercoagulability, venous thromboembolism, ascites).

The term “treating a glomerular disease” refers to ameliorating the effects of, or delaying, halting, or reversing the progress of, a glomerular disease as defined herein. In some embodiments, treating the glomerular disease can refer to the selection and administration of the appropriate therapy based on the type of glomerular disease present in the patient. For example, appropriate therapies for patients diagnosed with glomerular disease (including non-ICGN glomerular disease) include, e.g. standard renoprotective therapies such as angiotensin-converting enzyme (ACE) inhibitors (e.g., enalapril, benazepril, captopril, ramipril), angiotensin receptor blockers (ARBs; e.g., telmisartan, losartan), management of blood pressure (e.g., beta-blockers, calcium channel inhibitors), anticoagulants (e.g., aspirin, clopidogrel, rivaroxaban), omega-3 fatty acid supplementation, and specially formulated diets for renal disease patients (See, J Vet Intern Med. 2013. November-December; 27 Suppl 1: S27-43). Additionally, appropriate therapies for patients diagnosed with ICGN include administration of mycophenolate, cyclophosphamide, azathioprine, chlorambucil, and cyclosporine.

“Patient” as used herein includes, e.g., a dog or a cat. In preferred embodiments, the patient is a dog. A “dog” may include, e.g., a Labrador Retriever, Golden Retriever, German Shepherd, Beagle, French Bulldog, Bulldog, Poodle, Yorkshire Terrier, Boxer, Rottweiler, Pembroke Welsh Corgi, Cavalier King Charles Spaniel, Dachshund, Dobermann, Shih Tzu, Australian Shepherd, Boston Terrier, German Shorthaired Pointer, Great Dane, Pomeranian, Bernese Mountain Dog, Siberian Husky, Miniature Schnauzer, a Border Collie, or a combination thereof (i.e. mixed breeds).

As used herein, the term “control” dog (or likewise a “control” cat) refers to an appropriate dog or cat that is used for comparison to a patient. In some embodiments, a control dog lacks glomerular disease. In other embodiments, a control dog comprises a glomerular disease type different than the patient. For example, in specific embodiments a control dog may comprise amyloidosis or glomerulosclerosis, whereas the patient comprises immune complex-mediated glomerulonephritis.

In some embodiments, the present methods direct a clinical decision regarding whether a patient is to receive a specific treatment. In one embodiment, the present methods are predictive of a positive response to immunosuppressive therapy. In certain embodiments, the present invention directs the treatment of a glomerular disease patient, including, for example, what type of treatment should be administered or withheld. For example, a patient that is shown to have an elevated level of miR-126 relative to a control dog may receive such treatment as immunosuppressive therapy. In one embodiment, the present methods may indicate that a patient will not be or will be less responsive to a specific treatment and therefore such a patient may not receive such treatment as immunosuppressive therapy. Accordingly, in some embodiments, the present methods provide for providing or withholding immunosuppressive therapy according to a patient's likely response. In this way, a patient's quality of life may be improved and the cost of care may be reduced.

In some embodiments, the present methods direct a clinical decision regarding whether a patient is to receive a specific type of treatment. Accordingly, in some embodiments, the present methods are a guiding test for patient treatment. Furthermore, the present methods provide information about the likely response that a patient is to have to a particular treatment. In some embodiments, the present methods provide a high likelihood of response and may direct treatment. In some embodiments, the present methods provide a low likelihood of response and may direct cessation of treatment or avoidance of specific treatments, including immunosuppressive therapy, and the use of alternative renal protective therapies, to avoid unnecessary negative effects from contraindicated therapies for a better quality of life.

As used herein, the term “polynucleotide” or “nucleic acid” generally refer to a polymer of nucleotide units and includes reference to a deoxyribonucleotide or ribonucleotide polymer. Polynucleotides include both single-stranded and double-stranded nucleic acid molecules. Polynucleotides include short single-stranded nucleic acid molecules composed of 13 to 25 nucleotides. In some embodiments, the term may refer to a nucleic acid molecule composed of less than 13 nucleotides, including 6-mer, 7-mer, 8-mer, 9-mer, 10-mer, 11-mer and 12-mer, or more than 25 nucleotides.

The term “RNA” refers to ribonucleic acid, a molecule of RNA encoding for a protein product or non-coding for a protein product (such as miRNAs but not excluding other non-coding RNAs). In particular, microRNA (i.e. “miRNA” or “miR”) is a noncoding RNA consisting of about 22 ribonucleotides. MicroRNAs (i.e. miRNAs or miRs) are small, non-coding, highly conserved RNAs that post-transcriptionally regulate gene expression. They play essential roles in governing biological activity in health and disease. MicroRNAs are provided herein are diagnostic for glomerular disease type, glomerular disease progression, and/or fibrosis severity. This allows for methods for treating a patient, such as a dog or cat, by assaying for and quantifying glomerular disease-related miRNAs in a sample obtained from the animal in the absence of a renal biopsy. In some aspects, detecting and measuring the abundance of the miRNAs disclosed herein provides a diagnostic tool for treating the patient most effectively.

The term “level” or “expression level” refers, e.g., to a determined abundance or relative abundance of a nucleic acid of interest. The term “pattern of expression levels” refers to a determined level of expression compared either to a reference nucleic acid, e.g. from a control, or to a computed average expression value, e.g. in DNA-chip analyses. A pattern is not limited to the comparison of two nucleic acids but is also related to multiple comparisons to reference nucleic acids or samples. A certain “pattern of expression levels” may also result and be determined by comparison and measurement of several nucleic acids of interest disclosed herein and display the relative abundance of these transcripts to each other. Expression levels may also be assessed relative to expression in different patients versus healthy controls, etc.

As used herein, the term “stage of glomerular disease” refers to a qualitative or quantitative assessment of the level of advancement of a glomerular disease. Criteria used to determine the stage of a glomerular disease includes, but is not limited to, the amount of miRNA associated with glomerular disease in a urine sample from a patient.

As used herein “amplified” or “amplification” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence-based amplification (NASBA, Cangene, Mississauga, Ontario), Q-Beta Replicase systems, transcription-based amplification system (TAS), and strand displacement amplification (SDA). See, e.g., Diagnostic Molecular Microbiology. Principles and Applications, D. H. Persing et al., Ed., American Society for Microbiology, Washington, D.C. (1993). The product of amplification is termed an amplicon.

The terms “hybridization” or “anneal” refer to the process by which single strands of nucleic acid sequences form double-helical segments through hydrogen bonding between complementary nucleotides. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

The term “oligonucleotide” refers to a short polynucleotide sequence (usually 6 to 100 nucleotides) joined by phosphorous linkages (e.g., phosphodiester, alkyl and aryl-phosphate, phosphorothioate), or non-phosphorous linkages (e.g., peptide, sulfamate and others). An oligonucleotide may contain modified nucleotides having modified bases (e.g., 5-methyl cytosine) and modified sugar groups (e.g., 2′-O—methyl ribosyl, methoxyethyl ribosyl, 2′-fluoro ribosyl, 2′-amino ribosyl, and the like). Oligonucleotides may be naturally occurring or synthetic molecules of double- and single-stranded DNA and double- and single-stranded RNA with circular, branched, or linear shapes and optionally including domains capable of forming stable secondary structures (e.g., stem-and-loop and loop-stem-loop structures).

A “primer” is a DNA molecule that is designed for use in annealing or hybridization methods that involve an amplification reaction. An amplification reaction is an in vitro reaction that amplifies template nucleic acid to produce an amplicon. As used herein, an “amplicon” is a DNA molecule that has been synthesized using amplification techniques. Amplicons of the invention have a polynucleotide sequence comprising a DNA sequence complementary to any one of SEQ ID NOs: 1-7, or fragments thereof. A pair of primers may be used with template DNA, such as a sample of cDNA, in an amplification reaction, such as polymerase chain reaction (PCR), to produce an amplicon, where the amplicon produced would have a DNA sequence corresponding to sequence of the template DNA located between the two sites where the primers hybridized to the template. A primer is typically designed to hybridize to a complementary target DNA strand to form a hybrid between the primer and the target DNA strand. The presence of a primer is a point of recognition by a polymerase to begin extension of the primer using as a template the target DNA strand. Primer pairs refer to use of two primers binding opposite strands of a double stranded nucleotide segment for the purpose of amplifying the nucleotide segment between them. Primer pairs of the present invention may in certain embodiments also be defined as comprising a first and second DNA molecule, wherein each are of sufficient length to function as DNA primers when used together in an amplification reaction with DNA (e.g. cDNA) comprising a sequence corresponding to, e.g. miR-126, miR-128, miR-21, or miR-182 to produce an amplicon diagnostic for glomerular disease in a dog.

The term “probe” refers to a single-stranded oligonucleotide sequence that will recognize and form a hydrogen-bonded duplex with a complementary sequence in a target nucleic acid sequence analyte or its cDNA derivative. Probes according to the invention include not only deoxyribonucleic or ribonucleic acids but also polyamides and other probe materials that bind specifically to a target DNA sequence and the detection of such binding can be useful in detecting the presence or absence of the target polynucleotide sequence. A probe may be attached to a conventional detectable label or reporter molecule, such as a radioactive isotope, ligand, chemiluminescent agent, or enzyme.

Methods for designing and using primers and probes are well known in the art. Polynucleotide molecules comprising the full length of or fragments of SEQ ID NOs: 1-7 are useful as primers and probes for detecting the polynucleotides associated with glomerular disease described herein and can readily be designed by one of skill in the art using the sequences provided herein.

Probes and primers according to the invention may have complete sequence identity with the target sequence, although primers and probes differing from the target sequence that retain the ability to hybridize preferentially to target sequences may be designed by conventional methods. For a nucleic acid molecule to serve as a primer or probe it need only be sufficiently complementary in sequence to be able to form a stable double-stranded structure under the particular solvent and salt concentrations employed. Any conventional nucleic acid hybridization or amplification method can be used to detect in a sample the presence of an elevated level of the miRNA relative to a control dog that lacks glomerular disease. Probes and primers are generally at least about 11 nucleotides, at least about 18 nucleotides, at least about 24 nucleotides, or at least about 30 nucleotides or more in length. Such probes and primers hybridize specifically to a target DNA sequence under stringent hybridization conditions. Conventional stringency conditions are described by MR Green and J Sambrook, Molecular cloning: a laboratory manual, 4th Edition, Cold Spring Harbor Laboratory Press, Cold Spring Harbor, N.Y. (2012). As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Detection (e.g., of an amplification product, of a hybridization complex, of a polynucleotide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. Examples of detection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides.

The present invention provides a novel method of detecting glomerular disease in a dog comprising detecting in a sample the presence of an elevated level of the miRNA relative to a control dog that lacks glomerular disease, wherein the elevated level of the miRNA relative to the control is indicative of the type of glomerular disease in the dog (e.g. amyloidosis, glomerulosclerosis, or immune complex-mediated glomerulonephritis). The levels of these circulating miRNAs present unique information, which may be used to determine the type of glomerular disease present, the stage of disease, and the severity of fibrosis. Such information can directly inform proper treatment of glomerular disease in dogs absent a renal biopsy and comprehensive pathological evaluation. In some embodiments, the elevated miRNA comprises a sequence selected from the group consisting of: SEQ ID NO:1, SEQ ID NO:2, SEQ ID NO:3, SEQ ID NO:4, and SEQ ID NO:7; or a combination thereof.

According to some embodiments, an elevated level of miRNA in a sample from a dog with glomerular disease is provided that is increased by at least 1.5-fold, at least 2-fold, at least 3-fold, at least 4-fold, at least 5-fold, at least 6-fold, at least 7-fold, at least 8-fold, at least 9-fold, at least 10-fold, or more than 10-fold, relative to a control dog that lacks glomerular disease. According to other embodiments, a depressed level of miRNA in a sample from a dog with glomerular disease is provided that is decreased by at least 1.5-fold, at least 2-fold, at least 3-fold, at least 4-fold, at least 5-fold, at least 6-fold, at least 7-fold, at least 8-fold, at least 9-fold, at least 10-fold, or more than 10-fold, relative to a control dog.

Conventional methods for detecting miRNA include Northern blot analysis; hybridization-based detection using microarrays; a method of detecting and quantifying a certain miRNA by a two-step process comprising RT-PCR, which uses stem-loop primers binding complementarily to the miRNA, and subsequent quantitative PCR; and a method comprising tailing the 3′-end of miRNA with poly(A) using a poly(A) polymerase, synthesizing cDNA using a poly(T) adaptor as a primer, and then amplifying the miRNA using a miRNA-specific forward primer and a reverse primer based on the poly(T) adaptor.

A “PCR-based method” refers to methods comprising a polymerase chain reaction PCR. This is a method of exponentially amplifying nucleic acids, e.g. DNA or RNA by enzymatic replication in vitro using one, two or more primers. For RNA amplification, a reverse transcription may be used as a first step. PCR-based methods comprise kinetic or quantitative PCR (qPCR) which is particularly suited for the analysis of differential expression levels. When it comes to the determination of expression levels, a PCR-based method may for example be used to detect the presence of a given miRNA by (1) reverse transcription into cDNA with help of a reverse transcriptase enzyme, and (2) detecting the presence of a given cDNA with help of respective primers. This approach is commonly known as reverse transcriptase PCR (rtPCR). The term “PCR-based method” comprises both end-point PCR applications as well as kinetic/real time PCR techniques applying special fluorophores or intercalating dyes which emit fluorescent signals as a function of amplified target and allow monitoring and quantification of the target. Quantification methods could be either absolute by external standard curves or relative to a comparative internal standard.

When detecting glomerular disease specific nucleic acid sequences in the form of RNA, e.g. miRNA, it may be preferred that the RNA is transcribed into cDNA prior to the detection of the glomerular disease specific nucleic acid sequences therein. RNA can be reverse transcribed into cDNA using RNA-dependent DNA polymerases such as, for example, reverse transcriptases from viruses, retrotransposons, bacteria, etc. These can have RNase H activity, or reverse transcriptases can be used that are so mutated that the RNase H activity of the reverse transcriptase was restricted or is not present (e.g. MMLV-RT RNase H—). RNA-dependent DNA synthesis (reverse transcription) can also be carried by enzymes that show altered nucleic acid dependency through mutation or modified reaction conditions and thus obtain the function of the RNA-dependent DNA polymerase. Commercial kits are available to reverse transcribe RNA into cDNA.

Once the RNA is reverse transcribed into cDNA, the DNA sequence can be analyzed for the presence and type of glomerular disease using for instance selective nucleic acid hybridization as described above. Such techniques are well known in the art and may comprise selective amplification using specific amplification primers, which may be designed based on the glomerular disease specific sequences described herein, e.g. SEQ ID NOs: 1-4 and 7.

The term “marker” or “biomarker” refers to a biological molecule, e.g., a nucleic acid, whose presence or concentration can be detected and correlated with a known condition, such as a disease state, or with a clinical outcome, such as response to a treatment. In particular, the present disclosure provides a method of detecting glomerular disease in a dog comprising obtaining a sample from the dog that comprises at least a first miRNA associated with glomerular disease; detecting in the sample the presence of an elevated level of the miRNA relative to a control dog; wherein the elevated level of the miRNA relative to the control is indicative of glomerular disease in the dog. In certain embodiments, the glomerular disease comprises amyloidosis, glomerulosclerosis, or immune complex-mediated glomerulonephritis; and the elevated level of the miRNA relative to the control is indicative of amyloidosis, glomerulosclerosis, or immune complex-mediated glomerulonephritis.

A reference pattern of expression levels may, for example, be obtained by determining in at least one healthy patient the expression level of at least one miRNA selected from the group consisting of miR-126, miR-128, miR-21, and miR-182; or by determining the expression level of at least one control miRNA present in the sample. According to an aspect of the invention, the expression levels of at least 2, 3, 4, 5, 6, 7, 8, 9, 10, 11, or 12 miRNAs are determined to obtain a pattern of expression levels.

The present disclosure also provides a method of detecting the presence of a miRNA corresponding to glomerular disease in a sample, the method comprising: contacting the sample comprising miRNA with a reverse transcriptase, wherein said reverse transcriptase produces a cDNA sequence corresponding to the miRNA; and (b) performing a polynucleic acid amplification reaction, thereby producing an amplicon; and (c) detecting said amplicon, wherein said amplicon comprises a DNA sequence produced from SEQ ID NO:1-4, and 7. In further embodiments, the method further comprises quantifying the amplicon.

The present disclosure further provides a kit for performing the methods of the invention, said kit comprising means for determining in a sample from a patient, an expression level of at least one miRNA selected from the group consisting of miR-126, miR-182, miR-21, and miR-128. The means for determining the expression level of said at least one miRNA may comprise an oligonucleotide probe for detecting or amplifying said at least one miRNA, means for determining the expression level based on an array-based method, a PCR-based method, a sequencing-based method, or any other suitable means for determining the expression level. The kit may include a miRNA primer pair, and in certain embodiments may also comprise reagents for quantification of the mRNA.

All the essential materials and/or reagents required for detecting glomerular disease associated miRNA in a sample may be assembled together in such a kit. This generally will comprise a miRNA primer pair of interest in the practice of the present invention. Also included may be enzymes or other reagents suitable for amplification and/or quantification, and, for instance, buffers to provide the necessary reaction conditions. Such kits generally will comprise, in suitable means, distinct containers for any reagent or enzyme, or for performing a given step in the contemplated quantification assay.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” may refer to a sequence disclosed herein.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a polynucleotide molecule comprising a sequence that when optimally aligned to a reference sequence provided herein, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as being indicative of glomerular disease in a patient.

### EXAMPLES

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention.

### Example 1

Materials and Methods for Investigating Micro-RNAs Associated with Glomerular Diseases

### Animal Specimens

In the retrospective studies described herein, samples submitted to the International Veterinary Renal Pathology Service at the time of renal biopsy between 2008 and 2016 were used, along with samples from clinically healthy dogs. Archived serum and urine samples were from 18 dogs with glomerular disease (6 from each diagnostic category) and 6 clinically healthy dogs for RNA-seq. For qRT-PCR, archived urine was from 49 dogs [AMYL (n=14), GS (n=14), and ICGN (n=21)] and 13 clinically healthy dogs. For RNA-seq, all dogs in the ICGN category had membranous glomerulonephritis (MGN). For qRT-PCR, the ICGN category consisted of 12 dogs with MGN, 7 dogs with membranoproliferative glomerulonephritis (MPGN), and 2 dogs with mesangioproliferative glomerulonephritis.

For clinically healthy dogs, a physical examination, complete blood count, chemistry panel, urinalysis, and urine protein:creatinine ratio (UPC) were performed to confirm their health status. For dogs with glomerular diseases, the diagnostic category of glomerular disease was confirmed by comprehensive renal biopsy. Samples were selected based on age (>1 year), urine sediment within 2 weeks of biopsy without discoloration or cloudiness on gross examination and with <5 WBCs/high-power field, <100 RBCs/high-power field, and no bacteriuria, adequate sample volume [serum: ≥1 mL; urine: ≥3 mL (RNA-seq) or 1 mL (qRT-PCR)], and non-hemolyzed serum base on a hemolysis score.

Within each diagnostic category, CKD dogs were further divided into Stage A [serum creatinine (sCr)<1.4 mg/dL or appropriately low for the breed and with biopsy findings limited to minimal to mild tubulointerstitial fibrosis] and Stage B [sCr>1.4 mg/dL or sCr<1.4 mg/dL but inappropriately high for the breed and biopsy results demonstrating moderate tubulointerstitial fibrosis]. Tubulointerstitial fibrosis was evaluated by a board-certified anatomic pathologist, scoring 0 to 5, as previously described (See Hokamp J A, et al. Veterinary Clinical Pathology 2018; 47:425-434). Each diagnostic category had approximately equal numbers of dogs in each stage.

### Sample Processing and Storage

Urine and serum samples corresponding with the renal biopsy were centrifuged to separate supernatant per standard protocol and shipped overnight on wet ice by the submitting clinician and then stored at −80° C. until analysis (1-6 years). For clinically healthy dogs, uncoagulated blood was allowed to sit at room temperature for 30-60 minutes after collection, then centrifuged at 1500 g for 10 minutes at room temperature to separate serum. Urine was collected via cystocentesis, and urine remaining after the urinalysis was centrifuged at 1000 g for 10 minutes at 4° C. Serum and urine were aliquoted and stored at −80° C. for approximately 3 years until RNA isolation. For clinically healthy dogs, the Texas A&M University Institutional Animal Care and Use Committee (IACUC) approved the protocol (AUP 2012-021), and client consent was obtained.

### RNA Isolation

All serum samples were screened for hemolysis by measuring the A385 and A414, using the NanoDrop 2000 (Thermo Fisher Scientific, Wilmington, DE, USA) and the following formula: hemolysis score=A414-A385+lipemia correlation factor*A385.17 Incorporating A385 in the calculation was done to minimize the lipemic interference when measuring A414.17 Scores were compared with an in-house hemolysis score cutoff value generated from a set of 28 grossly non-hemolyzed, leftover clinical samples submitted to the Texas A&M University Veterinary Medical Teaching Hospital (data not shown).

Circulating RNA was isolated from serum by a modified protocol using the Direct-zol RNA Miniprep Kit (Zymo Research, Irvine, CA, USA). For each dog, 1 mL serum was first homogenized with 5 mL QIAzol Lysis Reagent (Qiagen, Germany). The mixture was vortexed, then incubated at room temperature for 5 minutes. Next, 1.2 mL chloroform was added, and lysates were vortexed and incubated at room temperature for 5 minutes, followed by 4° C. centrifugation at 13400 g for 15 minutes. After centrifugation, the upper aqueous phase was mixed with 4.8 mL 100% ethanol, added to a Zymo-Spin Column (Zymo Research, Irvine, CA, USA), and centrifuged at 12000 g for 30 seconds at room temperature. The spin column was then washed twice with Zymo RNA pre-wash buffer (Zymo Research, Irvine, CA, USA), once with Zymo RNA wash buffer (Zymo Research, Irvine, CA, USA), and once with 500 mL 80% ethanol, for a total of 4 washes. RNA was eluted with 25 mL 50° C. RNase-free water.

Urinary RNA was isolated from 3 mL (for RNA-seq) or 1 mL (for qRT-PCR) urine supernatant from each dog using the Qiagen exoRNeasy Serum/Plasma Maxi Kit (Qiagen, Germany). The manufacturer's protocol was followed up to the point of adding QIAzol Lysis Reagent (Qiagen, Germany). The subsequent steps were identical to the serum isolation protocol, except that the RNeasy MinElute Spin Columns (Qiagen, Germany) were washed 3 times: once with Buffer RWT (Qiagen, Germany) and twice with Buffer RPE (Qiagen, Germany). RNA was eluted with 25 mL 50° C. RNase-free water.

### Small RNA Sequencing and Data Analysis

RNA samples were measured using the Fragment Analyzer High Sensitivity RNA Analysis Kit (Advanced Analytical Technologies, Inc., Ankeny, IA, USA). The Texas A&M University Genomics and Bioinformatics Service laboratory generated a cDNA library using the NEXTflex Small RNA Library Prep Kit (Bio Scientific Corp, Austin, TX, USA). Under a 50 base-pair, single-end setting, all 48 cDNA libraries were multiplexed and sequenced in parallel on 3 lanes of a flow cell in an Illumina Genome Analyzer (HiSeq 2500v4) to minimize technical variation and ensure sufficient data output.

Pre-processing of raw reads (fastq files) included removal of the 3′ adapter sequence (TGGAATTCTCGGGTGCCAAGG), trimming of the first and last 4 bases from the adapter-clipped reads (as recommended by the manufacturer), filtering out reads fewer than 16 base-pairs to prevent false degraded RNA or adapter dimers, and removal of low-quality reads (quality score <30). Untrimmed raw reads were discarded as they were unlikely to be miRNAs based on read lengths. FASTX-Toolkit (version 0.0.14) was used to transform the fastq format into collapsed fasta files as proper inputs for CPSS 2.0 (http://114.214.166.79/cpss2.0/index.html).20 Default settings along with the canine genome (Canis familiaris, CanFam 3.1) and microRNA annotation in miRBase (release 21) were used for analysis. The DESeq2 package in R was used to identify DE miRNAs. For multiple testing, Wald test P-values were corrected to the false discovery rate (adjusted P-values) by the Benjamini-Hochberg procedure. An adjusted P-value <0.05 was set to select DE miRs robustly. NormFinder (updated January 2015) was applied to the read count table to identify candidate miRNAs for internal controls for qRT-PCR based on the RNA-seq data, and urinary miR-151 and miR-28 were identified as the most appropriate internal controls. These miRNAs were not identified as DE miRNAs in any given pair of comparisons in this study.

gRT-PCR and Data Analysis

Based on sequencing data and preliminary qRT-PCR results, urinary miR-126, miR-21, miR-182, and miR-486 were selected as target miRNAs, along with miR-151 and miR-28 as reference miRNAs. The miRNA primers were acquired through GeneGlobe (Qiagen, Germany). RNA was reverse transcribed into cDNA using the T100 Thermocycler (Bio-Rad Laboratories, California) and MiRCURY LNA RT Kit (Qiagen, Germany). The cDNA was diluted using RNAse-free water at a 1:15 ratio and incorporated in the 10 μL PCR reaction using the miRCURY LNA SYBR Green PCR kit (Qiagen, Germany). The Epmotion 6000 (Eppendorf, MA, USA) was used for plate set-up to decrease pipetting errors, replicate, and plate differences. The PCR was run on 384-well plates using the CFX384 Touch Real-Time PCR Detection System (Bio-Rad Laboratories, CA, USA). Each sample was run in triplicate to account for technical variability and inter-plate calibrators. A standard curve for each miRNA and controls (NRT, NTC, SYBR neg, and water) were also included. The mean expression among the triplicates was used for analysis. If there were inconsistencies in the amplification curves, primer dimers were ruled out by running the PCR products on 1% agarose gels.

The mean, standard deviation, coefficient of variation, and efficiency for each miRNA were evaluated, and qbase+ was used to normalize the expression of target miRNAs to the reference miRNAs, calibrate expression differences between plates, and remove samples that did not meet QC requirements. Samples with a Cq difference >1.0 between the technical replicates and samples from any plates with miRNA amplification efficiencies <80% were excluded from the analysis. Mann-Whitney tests and one-way ANOVA were used to determine significant differences in log 10 normalized mean expression between diagnostic categories (miR-126) and clinical stage (miR-21, miR-182, miR-486). Mann-Whitney pair-wise non-parametric tests were used to evaluate miRNAs with non-normal distributions, while one-way ANOVA was used to evaluate miRNAs with normal distribution. The diagnostic performance of miRNAs as biomarkers using qRT-PCR was evaluated with the Wilson/Brown receiver operating characteristic (ROC) curve analysis. Area under the curve (AUC), 95% confidence intervals (CI), and cut points were calculated using non-parametric ROC methods. AUCs were considered statistically significant when p<0.05.

The Shapiro-Wilk goodness of fit test assessed the normality of clinical parameters [age, sCr, UPC, urine specific gravity (USG), and fibrosis]. Clinical parameter differences among diagnostic categories and stages were performed using Mann-Whitney pair-wise non-parametric tests. Correlations between clinical parameters and all target miRs were evaluated using Spearman correlations with a p<0.05 considered significant. All analyses were performed in GraphPad Prism9 (GraphPad Software, San Diego, CA) except for the AUC analysis, which used STATA17 (StataCorp LLC, College Station, TX).

### Example 2

### Clinical Parameters for Each Glomerular Disease Category

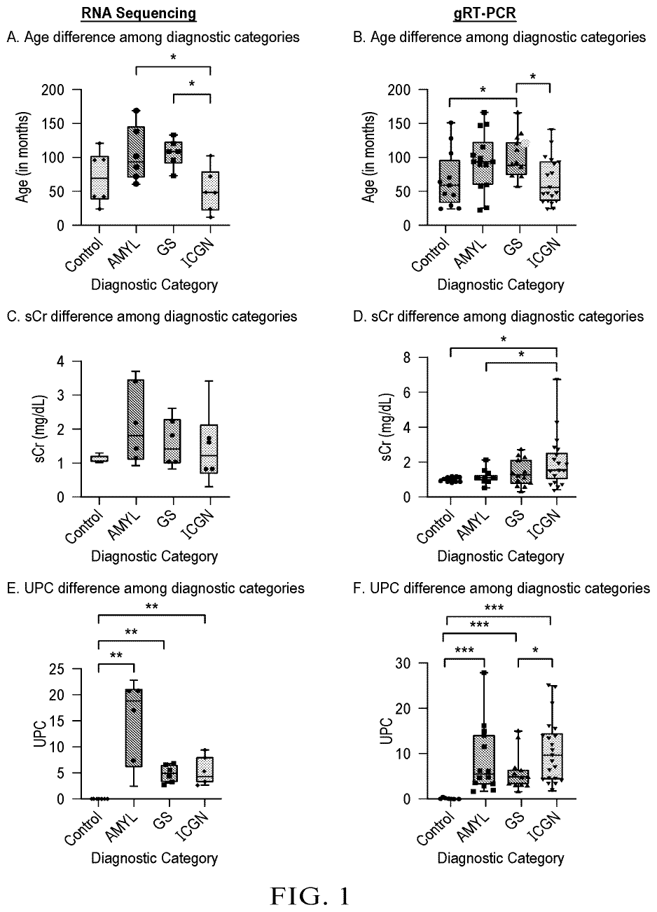

The median age was significantly lower for the ICGN category compared with AMYL and GS for dogs in the RNA-seq cohort and significantly lower compared with GS in the qRT-PCR cohorts. The median age of dogs in the GS category was significantly higher than controls in the qRT-PCR cohort (FIG. 1A, 1B). No significant difference in median sCr was observed among diagnostic categories for the RNA-seq cohort; however, in the qRT-PCR cohort, sCr was significantly higher in dogs with ICGN compared to dogs with AMYL and controls (FIG. 1C, 1D). UPC was significantly higher in dogs with ICGN compared to dogs with GS, and UPC was significantly higher in all diagnostic categories compared to controls (FIG. 1E, 1F). Based on the study design, sCr was significantly higher in stage B dogs than in stage A dogs and controls. UPC was also significantly higher in stage A and B dogs compared to controls. There was no difference in age between stage A and B dogs.

### Example 3

### Differentially Expressed (DE) Circulating and Urinary miRNAs

Forty-eight RNA samples were used for RNA-seq, 24 samples from serum and 24 from urine. One serum sample representing stage B ICGN was excluded for low reads (<5 million). Sequenced serum samples averaged 6.5 million reads and a 97.6% genome mapping rate, with 1.6 million reads mapped to miRNAs. Urine samples had an average of 6.9 million reads and a 90.1% genome mapping rate, with 79,019 reads mapped to miRNAs. On average, 167 and 88 miRNAs with at least 10 mapped reads were detected in serum and urine samples, respectively.

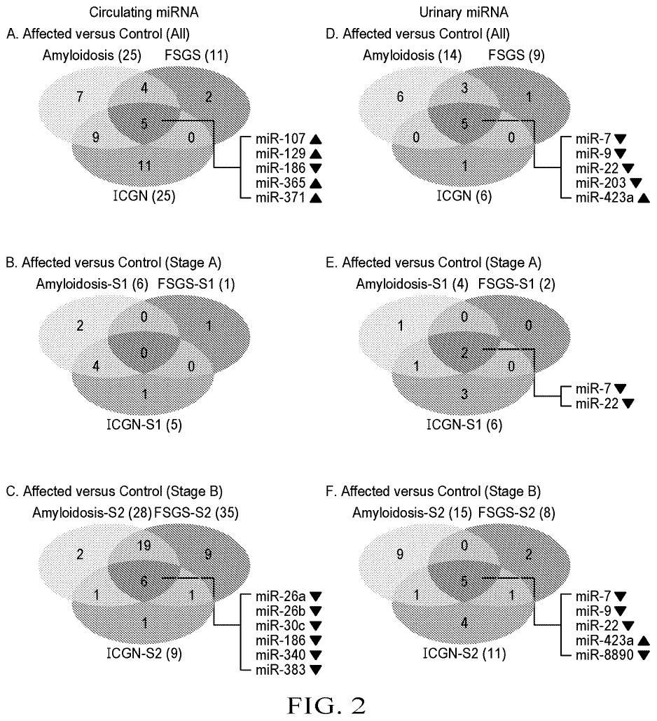

Overall, 38 circulating and 16 urinary miRNAs were DE in CKD dogs versus controls (FIG. 2). In each diagnostic category, CKD dogs were compared with controls regardless of disease stage (FIG. 2A, 2D). CKD dogs were further divided into stages A (FIGS. 2B, 2E) and B (FIG. 2C, 2F) and compared with controls. Comparing stage A dogs with controls, 8 circulating and 7 urinary DE miRNAs were detected, while 39 circulating and 22 urinary DE miRs were discovered, comparing stage B dogs with controls. Regardless of diagnostic category or stage, 5 circulating miRNAs (miR-107, miR-129, miR-186, miR-365, and miR-371) and 5 urinary miRNAs (miR-7, miR-9, miR-22, miR-203, and miR-423a) were DE in dogs with glomerular disease when compared with controls (FIG. 2A, D). Downregulated circulating miR-186 was also among the 6 common DE miRNAs among all 3 diagnostic categories when comparing stage B CKD dogs to controls (FIG. 2C). Downregulated urinary miR-7 and miR-22 were the only 2 common DE miRs among all 3 diagnostic categories identified in stage A dogs compared with controls. They also comprised 2 of the 5 DE miRs in stage B dogs compared with controls (FIG. 2E, 2F).

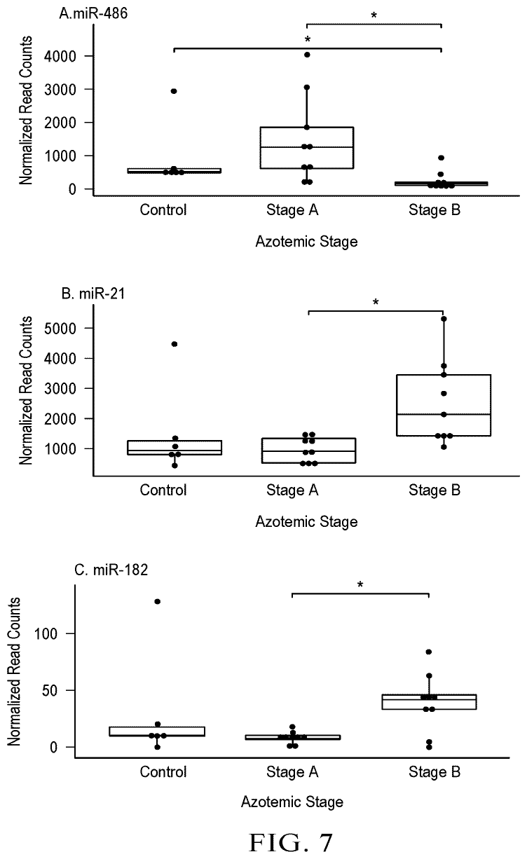

When all CKD dogs were combined, regardless of the glomerular diagnostic category, no circulating miRs were DE between stages A and B. However, 3 urinary DE miRs were identified between stage A and B dogs, including upregulated miR-182 and miR-21 and downregulated miR-486 (FIG. 7). Notably, the expression of urinary miR-486 was significantly decreased in stage B dogs compared with both stage A dogs and controls (FIG. 7).

### Example 4

### Differentially Expressed (DE) Glomerular Disease-Specific Urinary miRNAs

The following example describes the identification of biofluid-derived miRNAs differentially expressed in specific diagnostic categories of canine glomerular diseases. No circulating miRNAs were differentially expressed among the different diagnostic categories when including all dogs in each category, regardless of disease stage.

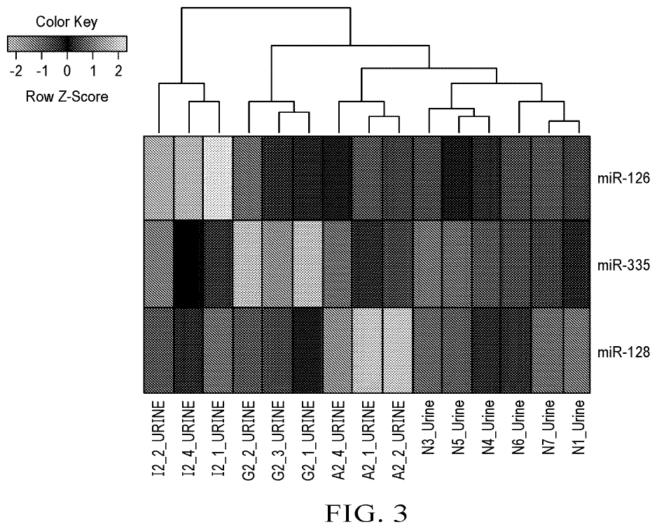

When comparing disease stages within each diagnostic category, an unlikely massive upregulation of circulating miR-1836 was noticed for AMYL in several comparisons (Table 1). The annotation of cfa-miR-1836 overlaps with snoRNA 20 (SNORA20) 23; therefore, miR-1836 was excluded from further analysis. For stage A, 3 circulating miRNAs (miR-335, miR-101, and miR-32) and 5 circulating miRNAs (miR-320, miR-99b, miR-218, miR-335, and miR-485) were differentially expressed in ICGN compared with AMYL and GS, respectively. No urinary DE miRs were identified among stage A samples. In stage B dogs, circulating miR-350 and miR-374a were differentially expressed in GS compared with AMYL and ICGN, respectively, while 5 urinary DE miRs were discovered in at least one pair of comparisons among the 3 glomerular diseases. Notably, the distinctive expression of urinary miR-126, miR-335, and miR-128 could correctly group stage B dogs into ICGN, GS, or AMYL (FIG. 3). This unique finding supports the idea that urinary miRNAs might help establish a diagnosis in azotemic dogs with suspected glomerular disease and indicate specific treatment options based on the same.

TABLE 1
Differentially Expressed (DE) miRs identified in each
glomerular disease category in Stage A and B Dogs.

| Stage A | | | Stage B | | |
|---|---|---|---|---|---|
| miRNA | Fold Change | Adjusted P-value | miRNA | Fold Change | Adjusted P-value |

Serum
AMYL vs. GS

| N/A | miR-1836* | −3726076 | 2.30E−05 |
|---|---|---|---|
| | miR-350 | 91.6 | 2.90E−02 |

AMYL vs. ICGN

| | | | | | |
|---|---|---|---|---|---|
| miR-335 | 86.7 | 1.10E−02 | miR-1836* | −5769001 | 1.80E−04 |
| miR-101 | 3.67 | 4.70E−02 | | | |
| miR-1836* | 23852 | 4.70E−02 | | | |
| miR-32 | 14.9 | 4.70E−02 | | | |

GS vs. ICGN

| | | | | | |
|---|---|---|---|---|---|
| miR-320 | −4.30 | 3.00E−03 | miR-374a | −5.86 | 2.03E−02 |
| miR-99b | −3.18 | 4.30E−02 | | | |
| miR-218 | 6.23 | 4.70E−02 | | | |
| miR-335 | 42.0 | 4.70E−02 | | | |
| miR-485 | −19.0 | 4.70E−02 | | | |

Urine
AMYL vs. GS

| N/A | miR-335 | −9.61 | 1.28E−02 |
|---|---|---|---|
| | miR-8904b | 20.8 | 1.28E−02 |

AMYL vs. ICGN

| N/A | miR-126 | −10.9 | 1.25E−02 |
|---|---|---|---|
| | miR-128 | 4.15 | 1.25E−02 |
| | miR-143 | −7.02 | 4.45E−02 |

GS vs. ICGN

| N/A | miR-8904b | −24.3 | 6.80E−03 |
|---|---|---|---|
| | miR-126 | −26.3 | 4.74E−02 |

qRT-PCR of urinary miR-126, miR-21, miR-182, and miR-486

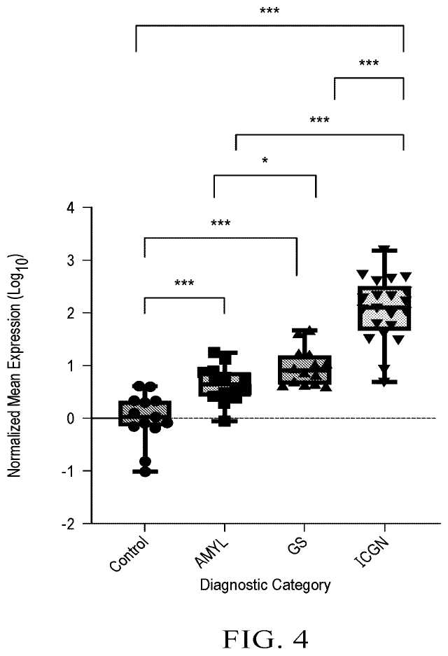

qRT-PCR confirmed that the normalized mean expression of urinary miR-126 was significantly higher in dogs with ICGN compared with AMYL, GS, and controls (FIG. 4), being 10.5 times higher than in dogs with GS (P<0.001) and 28.9 times higher compared to dogs with AMYL (P<0.001). Urinary miR-126 expression was also higher in dogs with GS compared to dogs with AMYL. No significant difference was observed within ICGN dogs between stage A and B or the ICGN subcategories (not shown). miR-126 expression was weakly to moderately correlated with sCr (r-0.330, P=0.008), UPC (r=0.493, P<0.001), and fibrosis (r=0.39, P=0.006; Table 2).

TABLE 2
Global Urinary MicroRNA Profiling of Canine Glomerular
Diseases Revealed Elevation of miRNA 126 in Dogs with
Immune Complex- Mediated Glomerulonephritis

| | | | |
|---|---|---|---|
| Clinical Parameter | r | p-value | Summary |

miR-126

| | | | |
|---|---|---|---|
| sCr | 0.33 | 0.008 | Weak, significant |
| UPC | 0.493 | 4E−05 | Moderate, significant |
| USG | 0.063 | 0.642 | Not significant |
| Fibrosis | 0.3926 | 0.006 | Moderate, significant |

miR-182

| | | | |
|---|---|---|---|
| sCr | −0.071 | 0.575 | Not significant |
| UPC | −0.2507 | 0.047 | Weak, significant |
| USG | 0.0339 | 0.803 | Not significant |
| Fibrosis | 0.2831 | 0.054 | Weak, significant |

miR-21

| | | | |
|---|---|---|---|
| sCr | 0.0919 | 0.47 | Not significant |
| UPC | 0.4166 | <.001 | Moderate, significant |
| USG | 0.1338 | 0.321 | Not significant |
| Fibrosis | 0.318 | 0.029 | Weak, significant |

miR-486

| | | | |
|---|---|---|---|
| sCr | 0.1394 | 0.272 | Not significant |
| UPC | −0.155 | 0.225 | Not significant |
| USG | 0.021 | 0.877 | Not significant |
| Fibrosis | 0.2592 | 0.078 | Not significant |

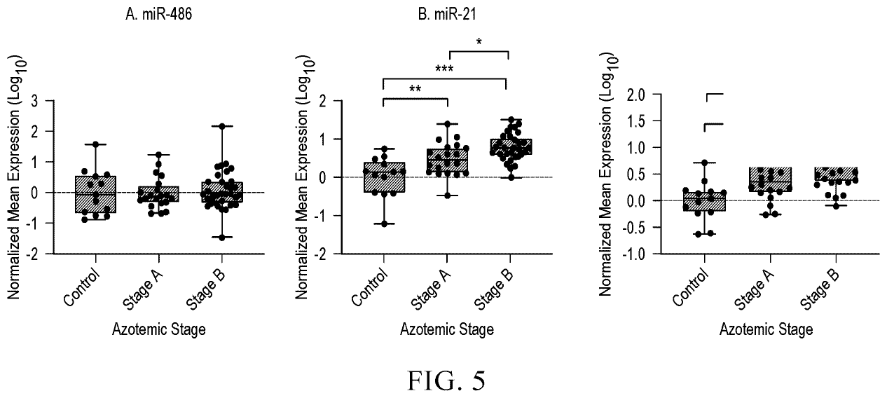

The normalized mean expression of urinary miR-21, miR-182, and miR-486 were evaluated for their ability to distinguish between stage A and B dogs and controls (FIG. 5). The normalized mean expression of miR-21 and miR-182 differed between stages and was 2.03 (P=0.009) and 2.15 (P=0.007) times higher, respectively, in stage B versus stage A dogs. Despite being differentially expressed based on RNA-seq, there was no difference in miR-486 between stage A and B dogs based on qRT-PCR (FIG. 5). MiR-21 was correlated with UPC (r-0.42, P<0.001) and with fibrosis (r-0.32, P=0.03; Table 2). Likewise, miR-182 was correlated with UPC (r=−0.25, P=0.05) and with fibrosis (r=0.28, P=0.05; Table 2). No significant correlations with clinical parameters were observed for miR-486. Fibrosis was weakly correlated with clinical stage (r=0.39, P=0.006; Table 2).

Diagnostic Evaluation of Urinary miR-126

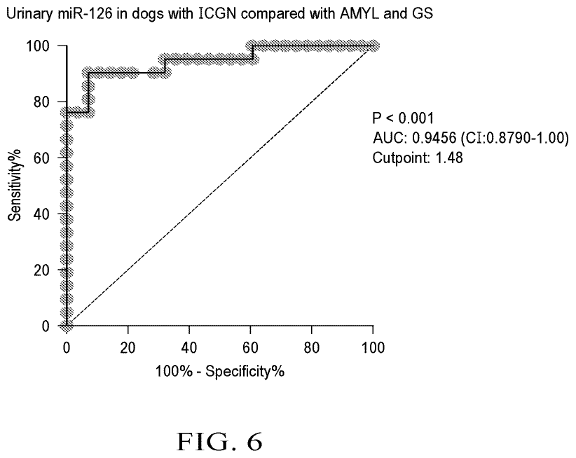

ROC curve analysis was performed to assess the ability of urinary miR-126 to distinguish between ICGN and non-ICGN (AMYL and GS) (FIG. 6). With a normalized mean expression and Log10 cut-point of 1.48, the AUC for miR-126 to differentiate dogs with ICGN versus non-ICGN was 0.9456 (CI: 0.88-1.0, P<0.001), with a sensitivity and specificity of 90% (CI: 71-98%) and 92% (CI: 77-99%), respectively.

MiR-126 had high sensitivity (90%) and specificity (92%) for ICGN compared with other glomerular diagnostic categories, correctly classifying 92% of cases. These findings support that urinary miR-126 can be used clinically as a non-invasive test to identify ICGN in dogs with suspected glomerular disease, using a normalized Log10 mean expression cut-point value ≥1.48. As ICGN is the most common category of glomerular disease in dogs biopsied for suspicion of glomerular disease and immunosuppressive therapy is typically recommended, a non-invasive test for ICGN would be particularly helpful for the effective treatment of ICGN in dogs.

### Example 5

### Additional Clinical Evaluation of Glomerular Disease-Associated miRNAs

Further experiments analogous to those described in Examples 1~4 will be carried out in dogs diagnosed with diseases that are common co-morbidities in dogs with renal disease. For dogs with non-renal diseases, diagnoses will be based on diagnostic tests (e.g., blood and urine testing, imaging, cytology/histology) performed by internal medicine and oncology specialists. Dogs with evidence of primary renal, ureteral, urethral, or bladder disease will be excluded (e.g., presence of azotemia, urolithiasis, or concerning changes on imaging). Additionally, samples from dogs with varying degrees of an active sediment and no clinical or historical evidence of renal disease will be selected from leftover samples submitted to the Texas A&M Veterinary Teaching Hospital Clinical Pathology Laboratory, and urine spiked with blood will be used to further assess interference by hematuria. Last, samples will be collected from clinically healthy dogs (determined by history, physical exam and laboratory testing).

It is anticipated that urinary miR-126 will demonstrate high sensitivity and specificity for ICGN with minimal influences by non-ICGN diseases. Alternatively, even if not specific for ICGN, these experiments would also be considered successful if miR-126 can identify diseases that would benefit from immunosuppression, since it is currently difficult to confidently identify many immune-mediated diseases.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit, and scope of the invention. All such variations and modifications apparent to those skilled in the art are deemed to be within the spirit, scope and concept of the invention as defined by the appended claims.

What is claimed is:

1. A method of detecting glomerular disease in a dog comprising:
(a) obtaining a sample from the dog that comprises at least a first miRNA associated with glomerular disease;
(b) detecting in the sample the presence of an elevated level of the miRNA relative to a control dog that lacks glomerular disease;
wherein the elevated level of the miRNA relative to the control is indicative of glomerular disease in the dog.

2. The method of claim 1, wherein the glomerular disease comprises amyloidosis, glomerulosclerosis, or immune complex-mediated glomerulonephritis.

3. The method of claim 1, wherein the sample is selected from the group consisting of serum, plasma, whole blood, and urine.

4. The method of claim 1, wherein the method comprises detecting in the sample an elevated level of at least a second miRNA.

5. The method of claim 1, wherein the miRNA is selected from the group consisting of:
miR-126, miR-128, miR-21, miR-182, and miR-335.

6. The method of claim 1, wherein the elevated level of the miRNA indicates the stage of glomerular disease progression.

7. The method of claim 6, wherein the elevated level of the miRNA further indicates fibrosis severity.

8. The method of claim 6, wherein the elevated level of the miRNA indicates that the dog has late stage glomerular disease.

9. The method of claim 1, wherein detecting in the sample the presence of an elevated level of the miRNA comprises RNA-Seq analysis or qRT-PCR analysis.

10. A method of treating a dog with glomerular disease comprising:
(a) obtaining a sample from the dog that comprises at least a first miRNA associated with glomerular disease;
(b) detecting in the sample the presence of an elevated level of the miRNA relative to a control dog that lacks glomerular disease; and
(c) treating the dog with glomerular disease based on the elevated level of the miRNA relative to the control.

11. The method of claim 10, wherein treating the dog comprises no treatment.

12. The method of claim 10, wherein treating the dog comprises immunosuppressive therapy.

13. The method of claim 10, wherein treating the dog does not comprise immunosuppressive therapy.

14. The method of claim 10, wherein treating the dog comprises administering a angiotensin-a converting enzyme (ACE) inhibitor, a angiotensin receptor blocker, an anticoagulant, an omega-3 fatty acid, a renal disease-specific diet, managing blood pressure, or a combination thereof.

15. The method of claim 12, wherein the immunosuppressive therapy comprises mycophenolate, cyclophosphamide, azathioprine, chlorambucil, or cyclosporine.

16. A method of detecting the presence of a miRNA corresponding to glomerular disease in a sample, the method comprising:
(a) contacting the sample comprising miRNA with a reverse transcriptase, wherein said reverse transcriptase produces a cDNA sequence corresponding to the miRNA; and
(b) performing a polynucleic acid amplification reaction, thereby producing an amplicon; and
(c) detecting said amplicon, wherein said amplicon comprises a DNA sequence produced from SEQ ID NO: 1-4 or 7.

17. The method of claim 17, wherein the method further comprises quantifying the amplicon.

18. A kit comprising:
(a) a miRNA primer pair;
(b) a means to quantitatively measure at least one miRNA; and
(c) a container for the substrate.

19. The kit of claim 18, wherein the miRNA primer pair quantitatively measures a miRNA selected from the group consisting of: miR-126, miR-128, miR-21, miR-182, miR-335.

20. The kit of claim 18, further comprising a substrate comprising a detectable label.

21. The kit of claim 20, wherein the detectable label is a fluorescent, radioactive, chromogenic, or chemiluminescent tag.


SEQUENCE LISTING (7 sequences)

SEQ ID NO: 1 - RNA, 21 nt, Canis lupus
  source 1..21: mol_type = unassigned RNA; sub_species = familiaris
cattattacttttggtacgcg

SEQ ID NO: 2 - RNA, 21 nt, Canis lupus
  source 1..21: mol_type = unassigned RNA; sub_species = familiaris
tcacagtgaaccggtctcttt

SEQ ID NO: 3 - RNA, 22 nt, Canis lupus
  source 1..22: mol_type = unassigned RNA; sub_species = familiaris
tagcttatcagactgatgttga

SEQ ID NO: 4 - RNA, 24 nt, Canis lupus
  source 1..24: mol_type = unassigned RNA; sub_species = familiaris
tttggcaatggtagaactcacact

SEQ ID NO: 5 - RNA, 21 nt, Canis lupus
  source 1..21: mol_type = unassigned RNA; sub_species = familiaris
tcgaggagctcacagtctagt

SEQ ID NO: 6 - RNA, 22 nt, Canis lupus
  source 1..22: mol_type = unassigned RNA; sub_species = familiaris
cactagattgtgagctcctgga

SEQ ID NO: 7 - RNA, 23 nt, Canis lupus
  source 1..23: mol_type = unassigned RNA; sub_species = familiaris
tcaagagcaataacgaaaaatgt